(19) United States
(12) Patent Application Publication
(10) Pub. No.: US 2026/0294575 A1
(43) Pub. Date: Oct. 1, 2026

(54) COVER FOR A MANUAL CONTROLLER OF A MEDICAL DEVICE

(71) Applicant: ALCON INC., Fribourg (CH)

(72) Inventors: Richard FERDON, Long Beach, CA (US); Brandon L. SPOELSTRA, Trabuco Canyon, CA (US); Brian T. CHIEM, San Gabriel, CA (US)

(21) Appl. No.: 19/567,093

(22) Filed: Mar. 14, 2026

Related U.S. Application Data
(60) Provisional application No. 63/780,550, filed on Mar. 31, 2025.

(51) Int. Cl. A61B 50/00; A61B 17/00

(52) U.S. Cl. CPC A61B 50/00; A61B 17/00; A61B 2017/00367; A61B 2017/0042; A61B 2017/0046

(57) ABSTRACT

In one or more embodiments, a handle cover for a handle of a medical device includes a grip portion and a shield portion. The grip portion receives the handle of the medical device and has a proximal end, a distal end, an inner surface, and an outer surface. The inner surface is shaped to receive to the handle. The inner surface forms an opening at the proximal end, where the opening is configured to receive the handle. The outer surface facilitates manual manipulation of the handle. The shield portion is coupled to the grip portion and is disposed between the grip portion and the medical device when the grip portion has received the handle.

### FIELD

Embodiments of the present disclosure relate to covers for manual controllers of medical devices.

### BACKGROUND

Medical devices may require a user to manually interact with a controller of the device in order to operate the device. For example, a user may manipulate a handle in order to move the device. As another example, a user may manipulate a joystick in order to input instructions to the device.

### SUMMARY

In one or more embodiments, a handle cover for a handle of a medical device includes a grip portion and a shield portion. The grip portion receives the handle of the medical device and has a proximal end, a distal end, an inner surface, and an outer surface. The inner surface is shaped to receive to the handle. The inner surface forms an opening at the proximal end, where the opening is configured to receive the handle. The outer surface facilitates manual manipulation of the handle. The shield portion is coupled to the grip portion and is disposed between the grip portion and the medical device when the grip portion has received the handle.

One or more embodiments may include none, one or more, and/or any combination of the following.

The grip portion includes one or more button pads, where a button pad allows for manual operation of a button of the handle.

The handle cover includes a cover catch that removably secures the handle cover to the handle.

The handle cover includes a cover tab that allows for manual separation of the handle cover from the handle.

The shield portion is coupled to the proximal end of the grip portion.

The shield portion is coupled to the distal end of the grip portion.

The length of the grip portion is within four centimeters of the length of the handle.

The length of the shield is within four centimeters of the length of the grip portion.

The handle cover comprises a silicone material.

In one or more embodiments, a method of utilizing a handle cover for a handle of a medical device includes placing a grip portion of the handle cover onto the handle for a first medical procedure. The grip portion has a proximal end, a distal end, an inner surface, and an outer surface. The inner surface is shaped to receive the handle. The inner surface forms an opening at the proximal end, where the opening receives the handle. The outer surface facilitates manual manipulation of the handle. A shield portion coupled to the grip portion is disposed between the grip portion and the medical device. The grip portion is removed from the handle to remove the handle cover from the medical device. The handle cover is sterilized. The grip portion is placed onto the handle of the medical device for a second medical procedure.

One or more embodiments may include none, one or more, and/or any combination of the following.

The grip portion is placed onto the handle by removably securing the handle cover to the handle using a cover catch of the handle cover.

The grip portion is removed from the handle by removing the grip portion using a cover tab of the handle cover.

In one or more embodiments, an objective housing cover for an objective housing of a delivery arm of a medical device includes a base and a wall. The base has a proximal end, a distal end, an upper surface, and an objective opening. The upper surface is shaped to conform to the objective housing. The objective opening is defined by the proximal end of the base and is shaped to receive the objective housing. The wall is disposed outwardly from the base and proximate to the distal end of the base. An inner surface of the wall is shaped to conform to the delivery arm.

One or more embodiments may include none, one or more, and/or any combination of the following.

The wall is substantially perpendicular to the base.

The base defines one or more light openings, where a light opening allows light from the delivery arm to pass through the base.

The distal end of the base has a cover tab that allows for manual removal of the objective housing cover from the delivery arm.

The inner surface of the wall has a cover catch that can couple with a delivery arm catch to removably couple the objective housing cover to the delivery arm.

The outer surface of the wall has a tubing management clip that couples tubing to the wall. The tubing management clip may include a slanted section, a middle section, and/or a secured section. The slanted section forms the opening of the tubing management clip and guides the tubing into the clip. The middle section has a clearance relative to the wall, where the clearance has a maximum size closer to the opening of the tubing management clip and a minimum size farther from the opening. The secured section allows the tubing to move in a direction along the axis of the tubing.

In one or more embodiments, a joystick cover for a joystick of a medical device includes a base, a wall, and a top. The base has an annular shape with an inner ring and an outer ring, where the inner ring is shaped to receive the joystick. The wall is disposed outwardly from the base and has a frustoconical shape or a cylindrical shape. The wall has an inner surface, an outer surface, a proximal end, and a distal end. The inner surface is shaped to receive the joystick. The outer surface has a texture that facilitates manual manipulation of the joystick. The proximal end of the wall is disposed outwardly from the inner ring of the base. The top is disposed outwardly from the distal end of the wall.

One or more embodiments may include none, one or more, and/or any combination of the following.

The difference between the diameter of the outer ring and the diameter of the inner ring is in a range of 0.5 and 3 centimeters.

The texture comprises a raised ridge, a rough area, and/or a high friction area.

The top has a button pad that allows for manual operation of a button of the joystick.

### BRIEF DESCRIPTION OF THE DRAWINGS

FIG. 1 illustrates examples of a handle cover and a control stick cover that may be used with an ophthalmic system, according to at least one embodiment described in the present disclosure;

FIG. 2 illustrates an example of a delivery arm and examples of covers for the manual controllers of the delivery arm, according to at least one embodiment described in the present disclosure;

FIG. 3 illustrates another example of a delivery arm and examples of covers for the manual controllers of the delivery arm, according to at least one embodiment described in the present disclosure;

FIGS. 4A-4C illustrate examples of handle covers that may be disposed onto a handle of a delivery arm, according to at least one embodiment described in the present disclosure;

FIGS. 5A and 5B illustrate an example of a joystick cover, according to at least one embodiment described in the present disclosure;

FIG. 6 illustrates an example of delivery arm on which an objective housing cover may be disposed, according to at least one embodiment described in the present disclosure;

FIGS. 7A and 7B illustrate views of an example of an objective housing cover, according to at least one embodiment described in the present disclosure;

FIGS. 8A and 8B illustrate views of another example of an objective housing cover, according to at least one embodiment described in the present disclosure;

FIGS. 9A-9C illustrate views of an example of an objective housing cover that includes a tubing management clip, according to at least one embodiment described in the present disclosure; and

FIG. 10 illustrates an example of a method of using a handle cover, according to at least one embodiment described in the present disclosure.

### DESCRIPTION OF EXAMPLE EMBODIMENTS

Referring now to the description and drawings, one or more example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Medical devices may require a user to manually interact with a controller of the device to operate the device. However, the manual interaction may lead to contamination of the controller, which may create an inconvenience or even a safety issue if the contamination occurs during a medical procedure. Accordingly, one or more embodiments described herein relates to a cover for a manual controller that may reduce contamination of the controller.

One or more embodiments of the present disclosure may provide improvements over previous iterations of covers for manual controllers. In one or more embodiments, a cover may serve as a sterile barrier that allows a user to move, operate, or otherwise handle a medical device without contaminating the device. For example, a cover for a handle of a delivery arm may allow a user to move the delivery arm without contaminating the handle. In one or more embodiments, a handle cover may have a shield portion that serves as a barrier to protect another part of the device in addition to the handle. In one or more embodiments, a cover may be reusable. For example, after using a cover for a medical procedure, the cover may be removed and sterilized for use in a subsequent medical procedure.

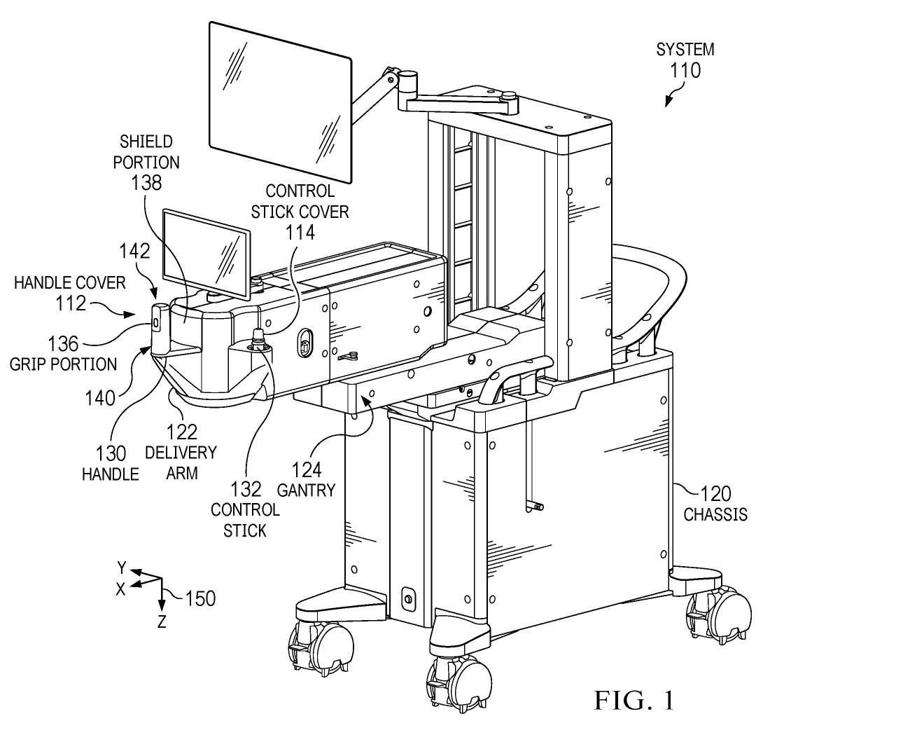

FIG. 1 illustrates examples of a handle cover 112 and a control stick cover 114 that may be used with an ophthalmic system 110, according to at least one embodiment described in the present disclosure. In the example, the ophthalmic system 110 includes a chassis 120, a delivery arm 122, and a gantry 124, which may be coupled as shown. The delivery arm 122 has a handle 130 and a control stick 132 (e.g., a joystick), which may be arranged as shown. The handle cover 112 has a grip portion 136 and a shield portion 138, which may be arranged as shown. The grip portion 136 has a proximal end 140 and a distal end 142.

As an overview of the example, the chassis 120 includes a laser source that generates a laser beam, and the delivery arm 122 has a laser head that directs the laser beam towards the eye of a patient. The gantry 124 facilitates movement of the delivery arm 122 relative to the chassis 120. The handle 130 of the delivery arm 122 may be used to manually move the delivery arm 122. The control stick 132 may be used to input instructions to a computer system of the ophthalmic system 110 to move the delivery arm 122 in an automated manner. The handle cover 112 may protect the handle 130 from contamination, and the control stick cover 114 may protect the control stick 132 from contamination, as described herein.

For ease of explanation, a chassis coordinate system 150 defined relative to the chassis 120 is shown. The chassis coordinate system 150 has an x-axis, a y-axis, and/or a z-axis, which may have any suitable relative angles among them. For example, the x-axis, the y-axis, and/or the z-axis may be orthogonal to each other. The z-axis may be aligned with or parallel to an optical axis of the ophthalmic system 110, and the x-axis and the y-axis may be orthogonal to the z-axis. As another example, an x-axis, a y-axis, and/or a z-axis may have other suitable angles (e.g., angles in the range of 1 to 179 degrees) relative to each other and may have any suitable geometric relationship with the ophthalmic system 110. Position in the chassis coordinate system 150 may be expressed using an x-coordinate, a y-coordinate, and/or a z-coordinate. Movement in an axial direction may be movement in a direction parallel to an axis. For example, movement in an x-direction may be movement in a direction parallel to the x-axis. Similarly, movement in a y-direction may be movement in a direction parallel to the y-axis, and movement in a z-direction may be movement in a direction parallel to the z-axis.

The ophthalmic system 110 may be any suitable system that may be used to perform a medical procedure (e.g., a surgical and/or diagnostic procedure) on an eye. Examples of ophthalmic diagnostic procedures include corneal topography, corneal keratometry, intraocular pressure measurement, tear film assessment, and/or eye tissue imaging procedures. Examples of ophthalmic surgical procedures include cataract, refractive, vitreoretinal, and/or other ophthalmic procedures. In one or more embodiments, the ophthalmic system 110 may be a cataract system that performs a cataract procedure.

Turning to the components of the ophthalmic system 110, the chassis 120 may support and/or house one or more components of the ophthalmic system 110, e.g., a laser source (not shown), the gantry 124, the delivery arm 122, and/or a computer system (not shown). The delivery arm 122 includes a laser head (not shown) that delivers a laser beam from the laser source. The gantry 124 facilitates movement of the delivery arm 122 in order to position the laser head. The delivery arm 122 may be moved from a stowed position to a deployed position. The stowed position may be, e.g., a retracted position such that the delivery arm 122 is closer to the chassis 120 and/or that all or a portion of the delivery arm 122 is at and/or within the chassis 120. The deployed position may be, e.g., an extended position such that the delivery arm 122 is ready to be aligned with the patient’s eye.

The ophthalmic system 110 may include one or more manual controllers. A user may use a manual controller to manually control the ophthalmic system 110 and/or manually input instructions to the ophthalmic system 110. For example, the user may use the handle 130 to manually move the delivery arm 122 or may use the control stick 132 to send instructions to a computer system to move the delivery arm 122 in an automated manner. The handle 130 may be any suitable size and/or shape, e.g., a size and/or shape suitable to be grasped by a human hand and/or one or more human fingers. In the example, the handle 130 may be grasped by a human hand to allows a user to manually move the delivery arm 122. As used herein, the term manual control may or may not include assistance by motive force. For example, the force to move the delivery arm 122 may be provided fully by the user’s grasping and moving the handle 130. As used herein, the term automated may include assistance by motive force with or without a user directing the movement. For example, a computer program may direct movement via one or more motors, or a user may direct the movement via one or more motors using the control stick 132.

A manual controller may have a cover. For example, the handle 130 may have the handle cover 112, and/or the control stick 132 may have the control stick cover 114. A cover for a manual controller may have any suitable size and/or shape. In one or more embodiments, the inner surface of the cover may have a size and shape that conform to the size and shape of the manual controller, while allowing for application of the cover to the manual controller and/or removal of the cover from the manual controller.

A cover for a manual controller may comprise any suitable material with any suitable characteristics, e.g., any suitable rigidness, flexibility, and/or sterilizability. In one or more embodiments, the material of the cover may be sufficiently rigid to be placed onto the manual controller and/or may be sufficiently flexible to be removed from the manual controller. In one or more embodiments, the material of the cover may be sterilizable, e.g., in an autoclave or in a disinfectant or other sterilizing agent. Examples of materials include silicone such as silicone rubber.

In one or more embodiments, a cover may include one or more features that facilitate use of the manual controller. In one or more embodiments, the cover may have a texture that allows a user to more readily manipulate the controller, e.g., the texture may provide friction that allows the user to manipulate the controller. The texture may include a raised ridge, a rough area, and/or other feature that provides friction. A raised ridge may be a raised geometrical shape, e.g., a rectangle (including a square), a polygon, and/or an oval (including a circle). A rough area may be a surface that is uneven and/or not smooth. Examples of texture are described herein.

In one or more embodiments, a cover may include an actuator pad that allows a user to manipulate an actuator of the controller. For example, a button pad may allow a user to press a button on the controller. An actuator pad may have any suitable size and/or shape, e.g., an actuator pad may have a size and/or shape that is substantially similar to the size and/or shape of the actuator. For example, a button pad may have a size and/or shape that is substantially similar to the size and/or shape of the button. An actuator pad may be at any suitable location of the cover, e.g., on a portion of the cover that is proximate to the actuator of the controller, e.g., outwardly from the actuator, when the cover is placed onto the controller in order to allow the user to manipulate the actuator. Examples of actuator pads are described herein.

In one or more embodiments, a cover may include a cover tab that allows a user to readily remove the cover from the controller. A cover tab may have any suitable size and/or shape. For example, a cover tab may be a protrusion that a user can grasp with, e.g., a thumb and a finger. A cover tab may have any suitable texture, e.g., a cover tab may have a texture as described herein. Examples of cover tabs are described herein.

In one or more embodiments, a cover may include a cover catch that removably couples the cover to the manual controller and may have any suitable size and/or shape. For example, an inner surface of the cover forms one or more protrusions, and an outer surface of the manual controller and/or other portion of the ophthalmic system 110 forms one or more indentation(s) that are sized and/or shaped to receive the protrusion(s). As another example, an outer surface of the manual controller and/or other portion of the ophthalmic system 110 forms one or more protrusion, and an inner surface of the cover forms one or more indentation(s) that are sized and/or shaped to receive the protrusion(s). Examples of cover catches are described herein.

In one or more embodiments, the handle cover 112 includes the grip portion 136 and the shield portion 138 coupled to the grip portion 136. The grip portion 136 receives the handle 130 and may protect the handle 130 from contamination from a user’s hand. The inner surface of the grip portion 136 may be shaped to conform to the handle 130. The inner surface forms an opening at the proximal end 140 through which to receive the handle 130. The outer surface of the grip portion 136 is configured to facilitate manual manipulation of the handle 130. For example, the outer surface may have a texture that allows a user to more readily manipulate the controller. When the grip portion 136 has received the handle 130, the shield portion 138 is disposed between the grip portion 136 and the delivery arm 122. The shield portion 138 may protect the delivery arm 122 from contamination from a user’s hand.

As an example of a method of utilizing the handle cover 112, the grip portion 136 is placed over the handle 130, e.g., before a medical procedure. The shield portion 138 is disposed between the grip portion 136 and the delivery arm 122. The handle 130 may be used to move the delivery arm 122, while the handle cover 112 prevents contamination of the handle 130 and the delivery arm 122. After the medical procedure has been completed, the grip portion 136 may be removed from the handle 130 to remove the handle cover 112 from the handle 130. The handle cover 112 is sterilized for another use. The grip portion 136 is placed over the handle 130, e.g., before a next medical procedure, to protect the handle 130.

The control stick 132 may comprise any suitable analog controller, e.g., a joystick. The control stick 132 may be any suitable size and/or shape, e.g., a size and/or shape suitable to be grasped by a human hand and/or one or more human fingers. In the example, the control stick 132 may be grasped by one or more human fingers to allow a user to send instructions to move the delivery arm 122 in an automated manner. The control stick cover 114 may protect the control stick 132 from contamination, as described in more detail herein.

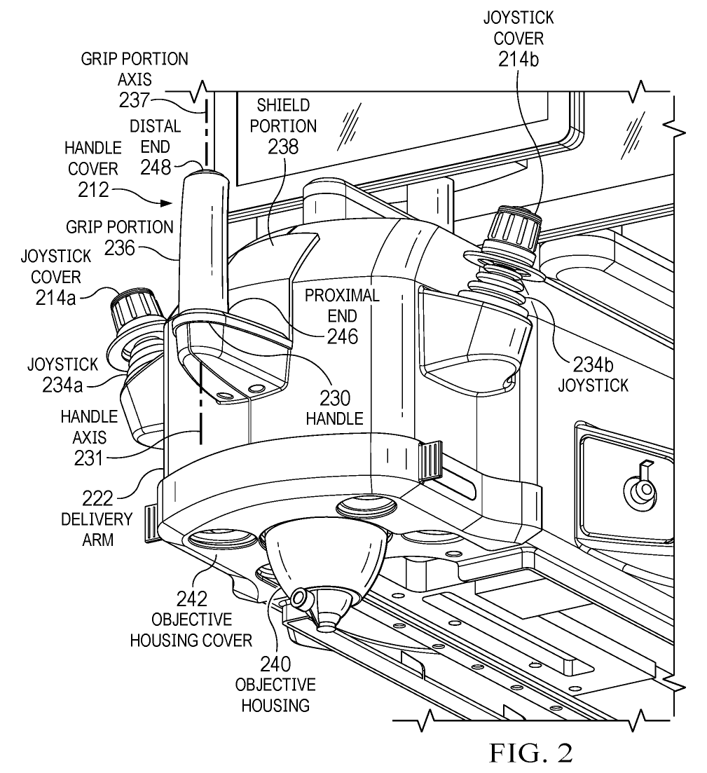

FIG. 2 illustrates an example of a delivery arm 222 and examples of covers for the manual controllers of the delivery arm 222, according to at least one embodiment described in the present disclosure. In the example, the delivery arm 222 includes a handle 230 (with a handle axis 231), joysticks 234 (234a and/or 234b), and/or an objective housing 240, which may be coupled as shown. The examples of covers include a handle cover 212, joystick covers 214 (214a and/or 214b), and an objective housing cover 242, which may be coupled as shown. The handle cover 212 (with a proximal end 246 and a distal end 248) includes a grip portion 236 (with a grip portion axis 237) and a shield portion 238, which may be coupled as shown. The handle cover 212 may have any suitable size and/or shape. The size and/or shape of the grip portion 236 may be selected relative to the size and/or shape, respectively, of the handle 230. For example, the length of the grip portion 236 (in the direction of the grip portion axis 237) may be within (i.e., greater than or less than) e.g., 0.1 to 0.5 centimeters (cm), 0.5 to 1 cm, 1 to 2 cm, 2 to 3 cm, and/or 3 to 4 cm of the length of the handle 230 (in the direction of the handle axis 231), and may be shorter than or longer than the length of the handle 230. As another example, the cross section (that is orthogonal to the grip portion axis 237) of the inner surface of the grip portion 236 may be substantially the same as or slightly larger than (e.g., 0.01 to 2 millimeters (mm) and/or 2 to 5 mm larger than) the cross section (that is orthogonal to the handle axis 231) of the handle 230.

The size and/or shape of the shield portion 238 may be selected relative to the size and/or shape, respectively, of the handle 230 and/or the average size of a user’s hand. For example, the length of the shield portion 238 (in the direction of the grip portion axis 237) may be within (i.e., greater than or less than) e.g., 0.1 to 0.5 centimeters (cm), 0.5 to 1 cm, 1 to 2 cm, 2 to 3 cm, and/or 3 to 4 cm of the length of the handle 230 (in the direction of the handle axis 231), or may be based on a breadth of a human hand (e.g., an average human hand size, an average human hand size plus an extra length portion, or a multiple (e.g., 1.2x) of an average human hand size). The shield portion 238 may have any suitable arrangement relative to the delivery arm 222. For example, the shield portion 238 may come in contact with a part of the delivery arm 222 (as shown). In such an embodiment, the shield portion 238 may follow a contour of the delivery arm 222. For example, as illustrated in FIG. 2, the shield portion 238 follows the curved contour of the delivery arm 222. In some embodiments, the shield portion 238 need not come in contact with a part of the delivery arm 222, e.g., the shield portion 238 may look like a sword guard. In such an embodiment, the shield portion 238 may or may not be in contact with the delivery arm for part of the shield portion 238, while another part is free-standing.

The shield portion 238 may be coupled to the grip portion 236 in any suitable manner. For example, the shield portion 238 may be coupled to proximal end 246 of the grip portion 236 (as shown). As another example, the shield portion 238 may be coupled to distal end 248 of the grip portion 236.

In one or more embodiments, any suitable covers for any suitable manual controllers may be packaged as a set of manual controller covers. The set may be packaged for, e.g., organizing, transporting, and/or selling the covers. The set may include any suitable manual controller covers, e.g., one or more handle covers 212, one or more joystick covers 214, one or more objective housing covers 242, and/or one or more other manual controller covers. For example, a set may include a handle cover 212, at least one joystick cover 214, and an objective housing cover 242. As another example, a set may include at least one handle cover 212, at least two joystick covers 214, and at least one objective housing cover 242. As another example, a set may include at least one handle cover 212 and at least two joystick covers 214. As yet another example, a set may include at least one handle cover 212 and at least one objective housing cover 242.

In the embodiments, the set may be packaged in any suitable container configured to hold the manual controller covers of the set. The container may comprise one or more components made from any suitable material. For example, the container may have a receptacle and a lid. Either or both parts may be made with a rigid and/or flexible material such as a polymer (e.g., plastic), a natural and/or synthetic fiber product (e.g., paper), and/or other suitable material. The receptacle may be shaped and sized to hold the set of manual controller covers. In one or more cases, the receptacle may include one or more compartments, where a compartment is sized and shaped to hold one or more manual controller covers. The lid may be removably coupled to the receptacle. In one or more cases, the lid may be coupled such that a user can readily remove the lid to access the manual controller covers in the receptacle.

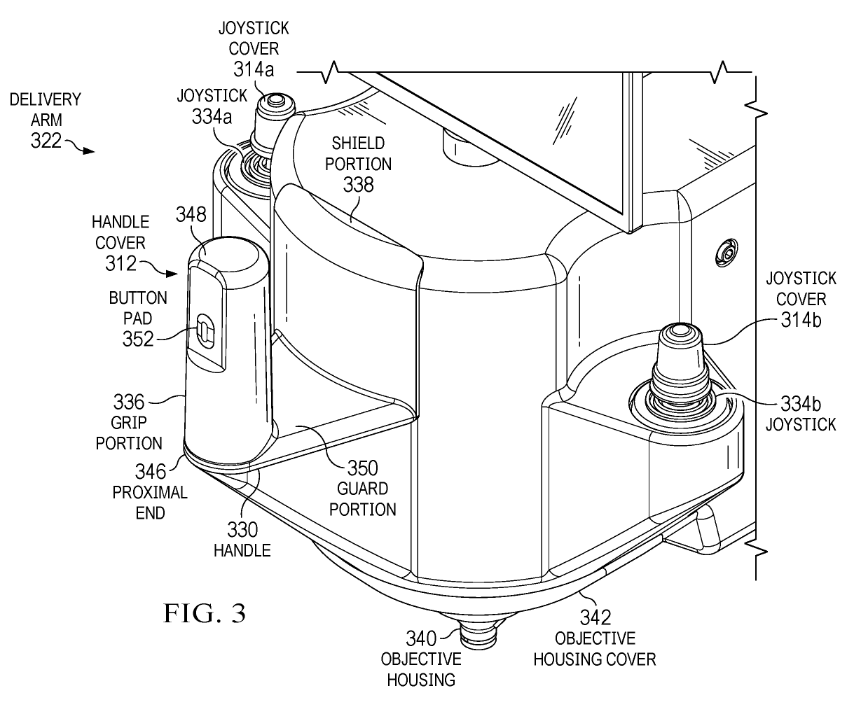

FIG. 3 illustrates an example of a delivery arm 322 and examples of covers for the manual controllers of the delivery arm 322, according to at least one embodiment described in the present disclosure. In the example, the delivery arm 322 includes a handle 330, joysticks 334 (334a and/or 334b), and/or an objective housing 340, which may be coupled as shown. The examples of covers include a handle cover 312, joystick covers 314 (314a and/or 234b), and an objective housing cover 342, which may be coupled as shown. The handle cover 312 (with a proximal end 346 and a distal end 348) includes a grip portion 336, a shield portion 338, and/or a guard portion 350, which may be coupled as shown. The grip portion includes a button pad 352.

The guard portion 350 of the handle cover 312 couples the grip portion 336 and the shield portion 338 and may protect the portion of the delivery arm 322 that couples the handle 330 to the rest of the delivery arm 322. The guard portion 350 may couple the grip portion 336 and the shield portion 338 in any suitable manner. For example, the guard portion 350 may couple the grip portion 336 and the shield portion 338 at the proximal end 346 of the handle cover 312 (as shown), at the distal end 348 of the handle cover 312, and/or at any other suitable part of the handle cover 312.

The grip portion 336 may include one or more button pads 352 that allow for manual operation of one or more buttons of the handle 330. A button pad 352 may be as described herein. In the example, the button pad 352 is located outwardly from a button (not shown) located on the handle 330 to allow a user to manually operate the button using the button pad 352.

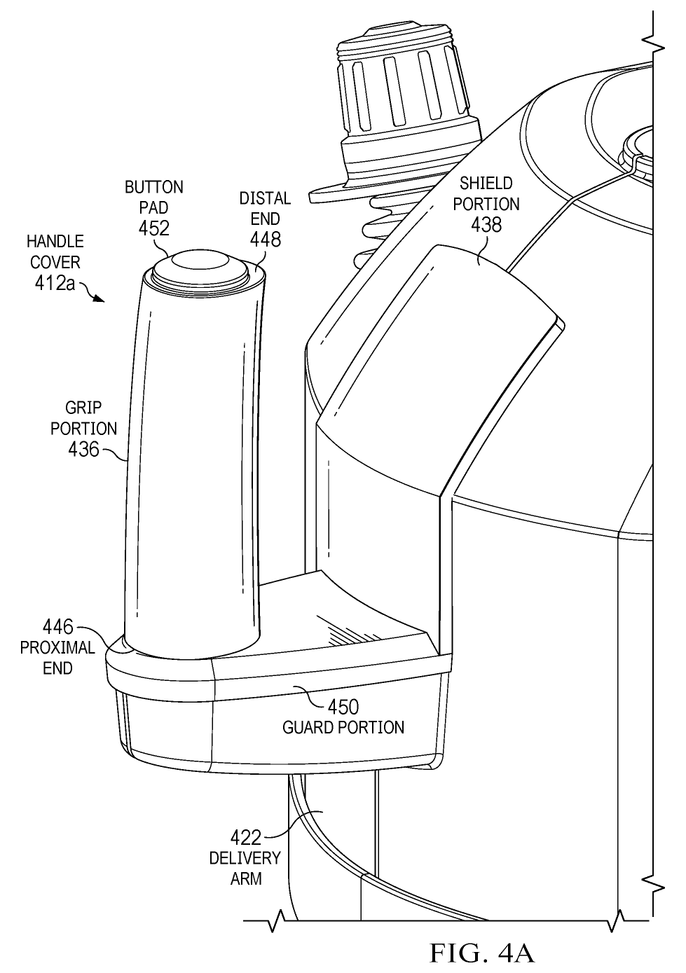

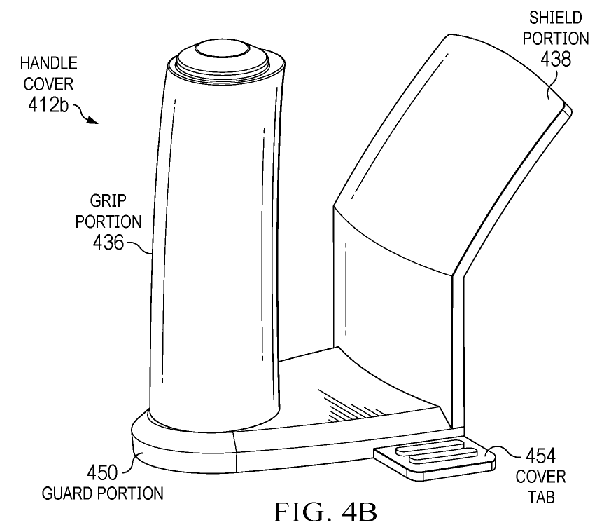

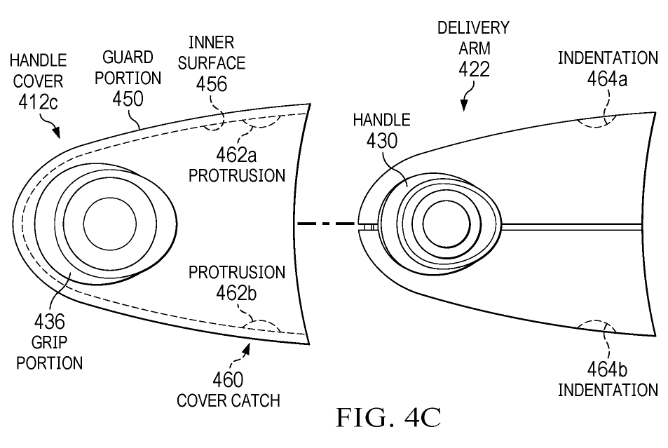

FIGS. 4A-4C illustrate examples of handle covers 412 (412a, 412b, and 412c, respectively) that may be disposed onto a handle of a delivery arm 422, according to at least one embodiment described in the present disclosure. FIG. 4A illustrates the handle cover 412a. In the example, the handle cover 412a (with a proximal end 446 and a distal end 448) includes a grip portion 436, a shield portion 438, and/or a guard portion 450, which may be coupled as shown. The grip portion includes a button pad 452 at the distal end 448.

FIGS. 4B and 4C illustrate example features of the handle covers 412b and 412c, respectively. FIG. 4B illustrates a cover tab 454 of the handle cover 412. The cover tab 454 may have any suitable size and/or shape and may be as described herein. In the example, the cover tab 454 is a protrusion that facilitates handling of the handle cover 412b. For example, the cover tab 454 can make it easier for a user to pull the handle cover 412b down over the handle (not illustrated in FIG. 4B) by providing a location to pull on the handle cover 412b. As another example, the cover tab 454 may make it easier for manual separation of the handle cover 412 from the handle of the delivery arm 422, for example, upon completion of a procedure.

In one or more embodiments, the handle cover 412b may have one or more cover tabs 454. For example, the handle cover 412b may have the cover tab 454 as shown. As another example, the handle cover 412b may have the cover tab 454 as shown and another cover tab (not shown), which may be located on the other side of the guard portion 450 from the cover tab 454 that is shown.

FIG. 4C illustrates a cover catch 460 of the handle cover 412c. The cover catch 460 removably couples (e.g., latches or interlocks) the handle cover 412c to the handle 430 of the delivery arm 422. The cover catch 460 may have any suitable size and/or shape and may be as described herein. In the example, an inner surface 456 of the guard portion 450 forms one or more protrusion(s) 462 (462a and/or 462b), and an outer surface of the delivery arm 422 forms one or more indentation(s) 464 (464a and/or 464b). The protrusion(s) 462 (462a and/or 462b) and the indentation(s) 464 (446a and/or 464b) may have any suitable size and/or shape. In the example, the protrusion(s) 462 (462a and/or 462b) and the indentation(s) 464 (464a and/or 464b) are sized and shaped such that an indentation 464 (464a or 464b) can receive a protrusion 462 (462a or 462b, respectively) to removably couple the handle cover 412 to the handle of the delivery arm 422. For example, the protrusion(s) 462 (462a and/or 462b) may interlock with or latch into the indentation(s) 464 (464a and/or 464b). In these and other embodiments, the protrusion(s) 462 (462a and/or 462b) and the indentation(s) 464 (464a and/or 464b) may be sized such that an interference fit between the protrusion(s) 462 (462a and/or 462b) and the indentation(s) 464 (464a and/or 464b) may prevent or resist at least some directions of movement of the handle cover 412c relative to the delivery arm 422. In some embodiments (not shown), the guard portion 450 may have one or more indentation(s) that can receive one or more protrusions of the delivery arm 422.

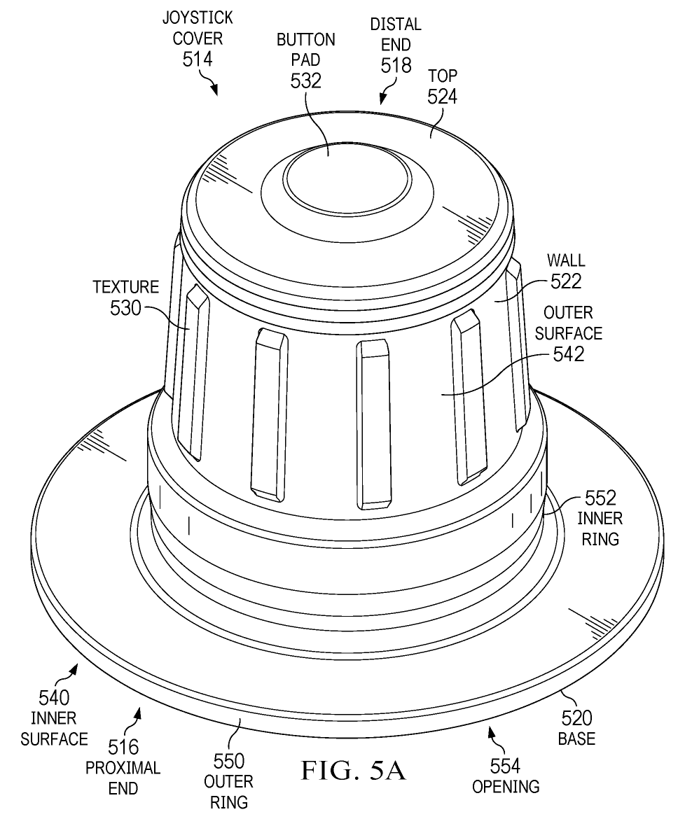

FIGS. 5A and 5B illustrate an example of a joystick cover 514, according to at least one embodiment described in the present disclosure. In the example, the joystick cover 514 has a proximal end 516 and a distal end 518 and includes a base 520 (at the proximal end 516), a wall 533, and a top 524 (at the distal end 518), which may be coupled as shown. The base 520 has a substantially annular shape with an outer ring 550 and an inner ring 552. The outer ring 550 has an outer diameter 560, and the inner ring 552 has an inner diameter 562. The width 564 of the annulus may be defined as the difference between the outer diameter 560 and the inner diameter 562. The wall 522 has an inner surface 540, an outer surface 542, and a texture 530. The top 524 has a button pad 532. As an example of using the joystick cover 514, the base has an opening (not shown) that is placed over the joystick until the distal end of the joystick reaches the inner surface of the top 524.

Turning to the components of the joystick cover 514, the base 520 fits over the joystick and may protect the joystick from contamination. The base 520 may have any suitable size and/or shape. In one or more embodiments, the opening 554 of the base 520 may be sized and shaped to accommodate the joystick. The width 564 of the annulus may have any suitable value. In the example, the width may be sufficiently small to allow for movement of the joystick and sufficiently large to prevent a user’s fingers from touching the joystick, e.g., the width 564 may be in a range of 0.5 to 3 centimeters (cm), such as 0.5 to 1 cm, 1 to 2 cm, and/or 2 to 3 cm.

The wall 522 fits over the joystick to protect the joystick from contamination. The wall 522 is disposed outwardly from the base 520, e.g., the proximal end 516 of the wall 522 may be coupled to the inner ring 552 of the base 520. The wall 522 may have any suitable size and/or shape. In one or more embodiments, the wall 522 may have a frustoconical shape or a cylindrical shape with the inner surface 540 and the outer surface 542. The difference between the diameter at the distal end 518 and the diameter at the proximal end 516 may be zero to 2 centimeters, such as 0 to 1 cm and/or 1 to 2 cm. In one or more embodiments, the diameter at the proximal end 516 of the wall 522 may be substantially equivalent to the inner diameter 562 of the base 520 where the wall 522 is coupled to the inner ring 552 of the base 520. In one or more embodiments, the wall 522 may have a height that accommodates the joystick such that the distal end of the joystick touches the inner surface of the top 524 when the base 520 rests against the delivery arm. The outer surface 542 has a texture 530, which may be as described herein. In the illustrated example, the texture 530 includes multiple raised ridges extending radially outward from the wall 522.

The top 524 is disposed outwardly from the wall 522, e.g., the top 524 may be coupled to the distal end 518 of the wall 522. The top 524 may have any suitable size and/or shape. In the illustrated example, the top 524 may have an outer diameter that matches the diameter of the distal diameter of the wall 522, e.g., is substantially equivalent to the distal diameter of the wall 522. The top 524 has a button pad 532, which may be as described herein. In the illustrated example, the button pad 532 is centrally located on the top 524, outwardly from a button (not shown) located on the joystick to allow a user to manually operate the button by pressing the button pad 532.

In some embodiments, the joystick cover 514 may include a guard portion and a shield portion in a similar or comparable manner to that described herein for the handle cover. For example, the base 520 may extend (e.g., have a diameter that is sufficiently large) to form a guard portion that protects a surface of an ophthalmic system (e.g., a surface of a delivery arm of the ophthalmic system) from contact with a user’s hand (e.g., a user’s finger). A shield portion may extend from the guard portion to further protect the surface of the ophthalmic system. The shield portion may extend in any suitable direction, e.g., in a distal direction away from the surface, in a direction along the surface, or in a direction between the distal direction and the direction along the surface. For example, the base 520 may extend towards a surface of the delivery arm as the guard portion, and an additional region of the joystick cover 514 may extend outward from the guard portion, and may or may not abut a surface of the delivery arm proximate the joystick.

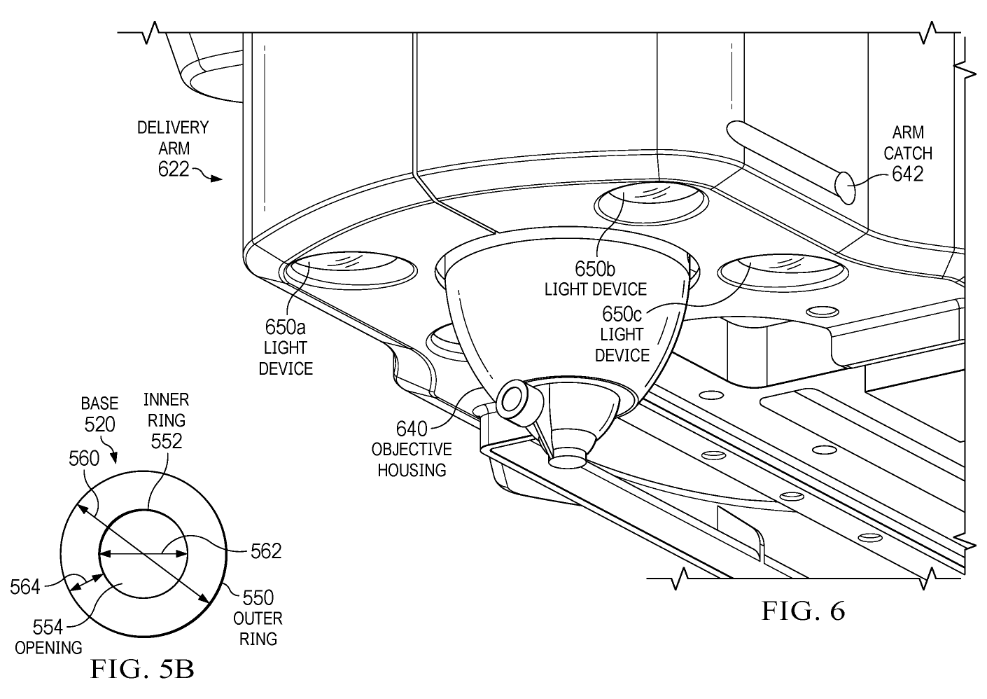

FIGS. 6-9C illustrate examples of objective housing covers that may be disposed about an objective housing of a delivery arm, according to at least one embodiment described in the present disclosure.

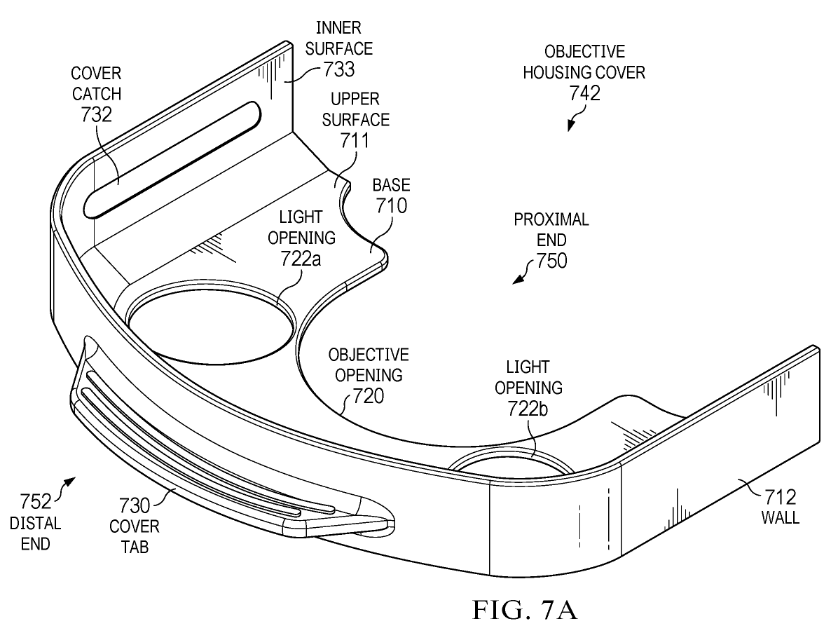

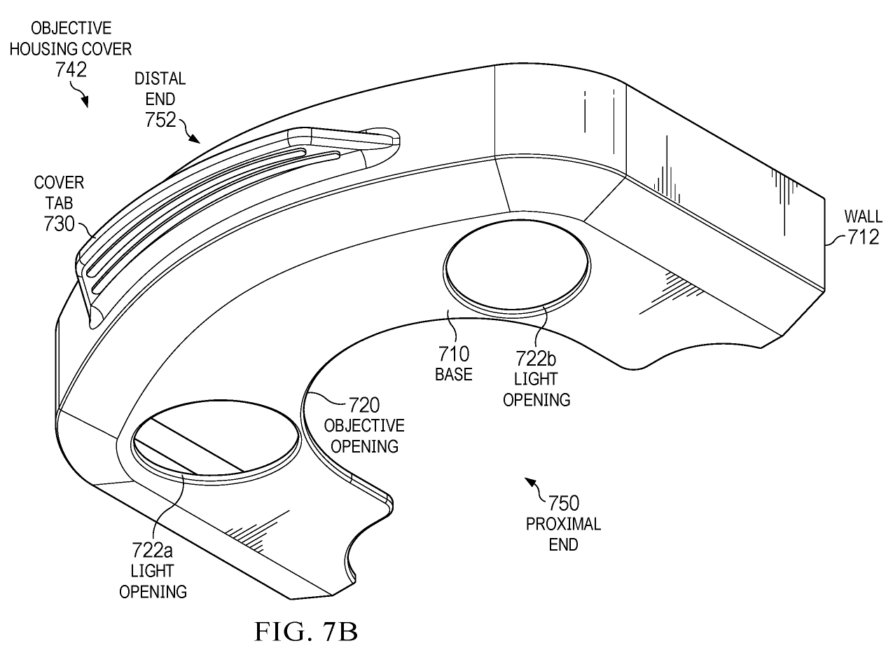

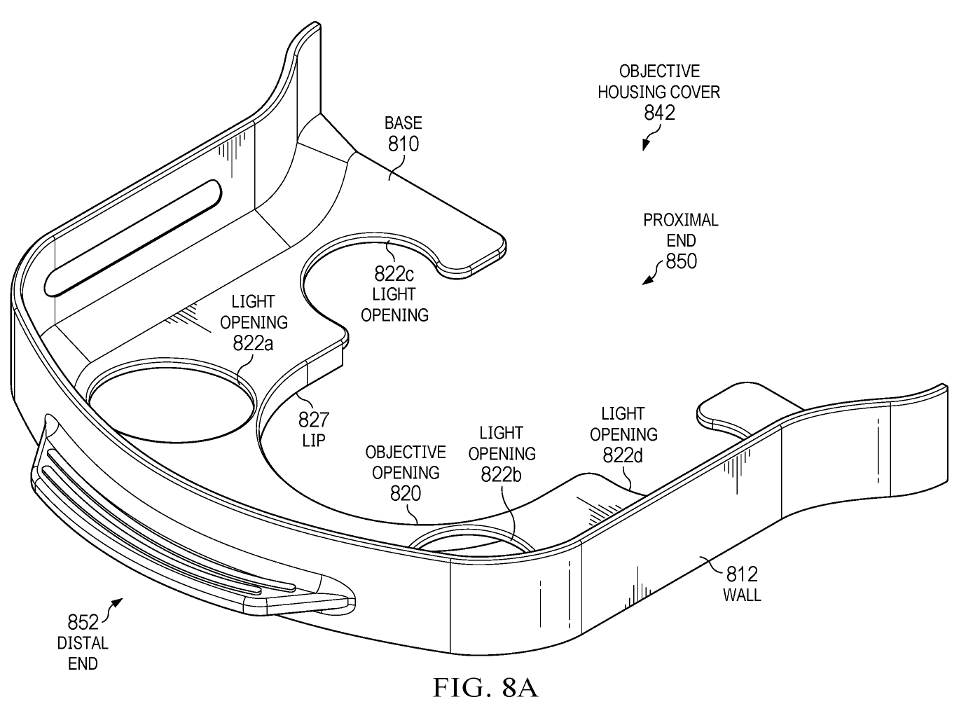

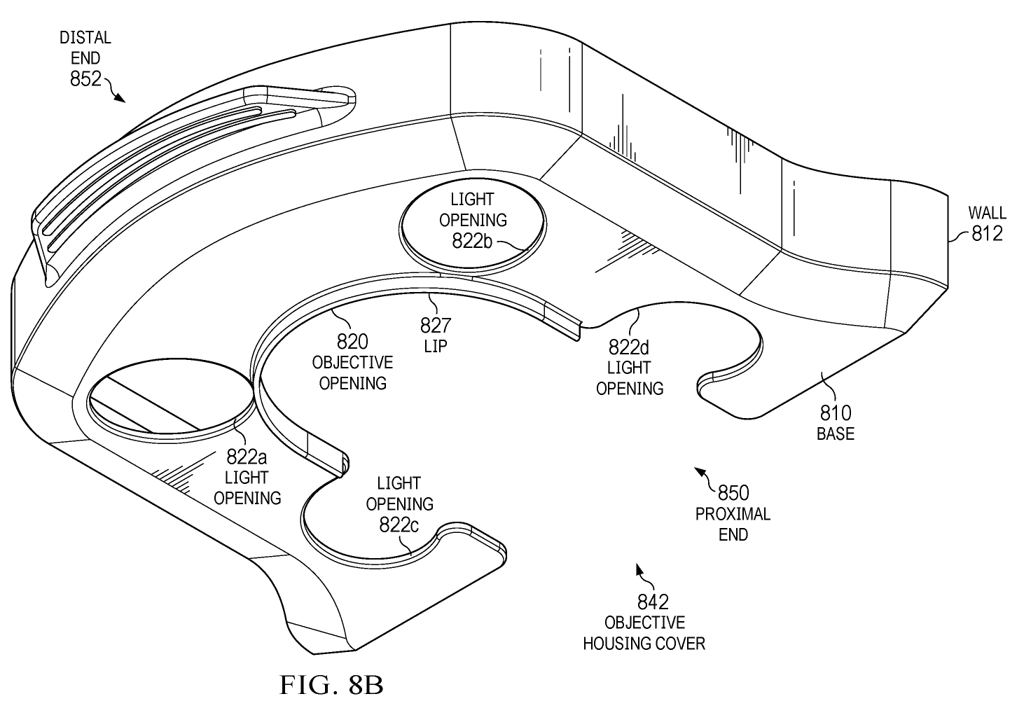

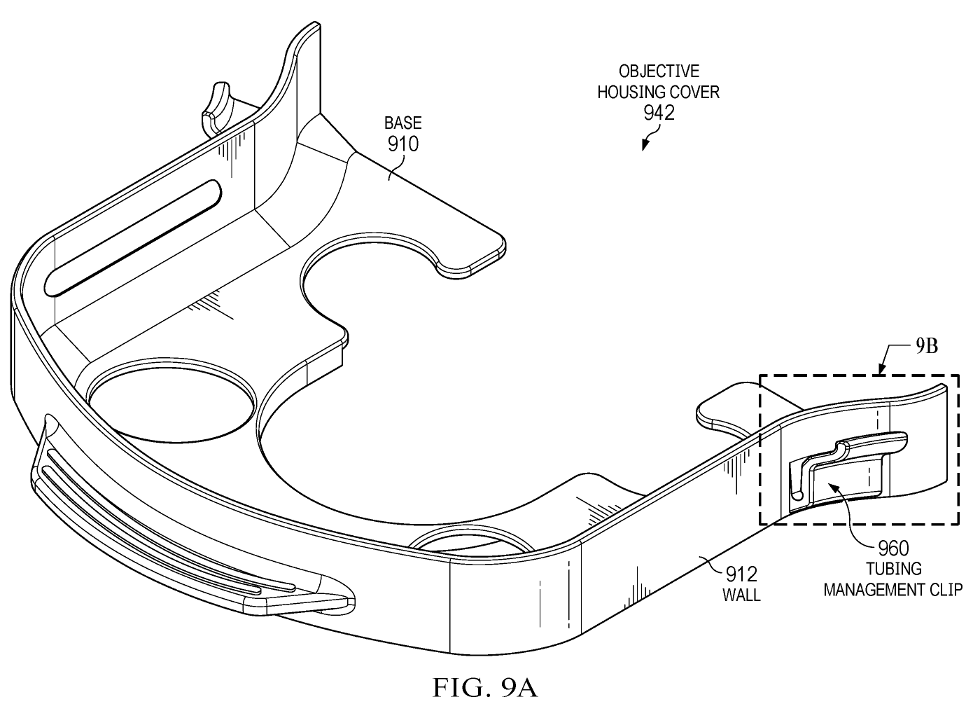

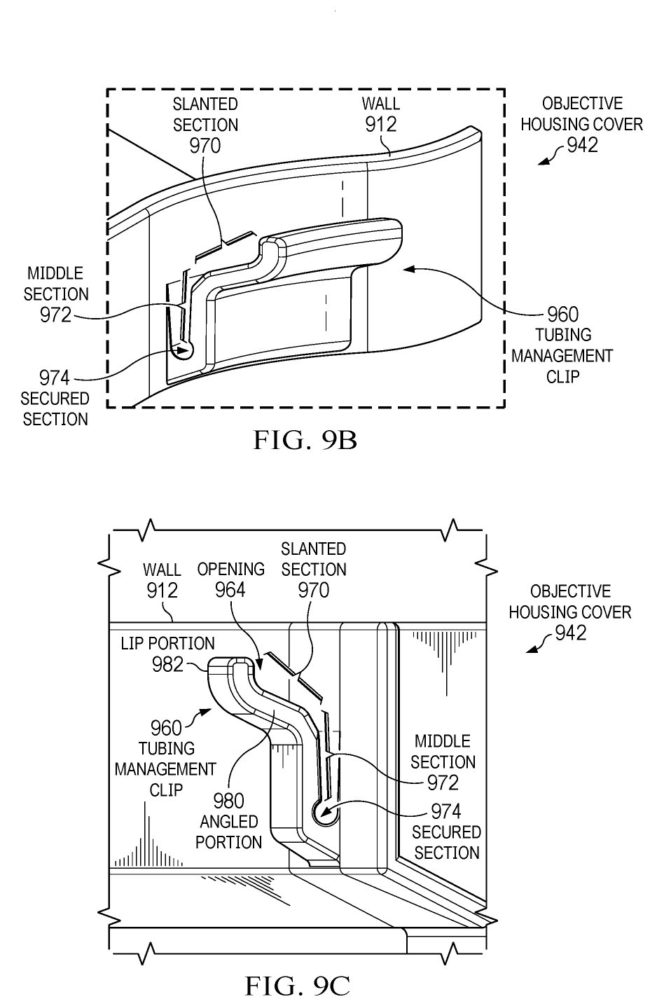

FIG. 6 illustrates the delivery arm 622 that includes the objective housing 640, an arm catch 642, and one or more light device(s) 650 (650a to 650c). The delivery arm 622 is illustrated without an objective housing cover attached. The objective housing 640 houses an objective lens of a laser head of the delivery arm 622. The arm catch 642 removably couples an objective housing cover (e.g., 742 of FIGS. 7A and 7B,842 of FIGS. 8A and 8B, or 942 of FIGS. 9A-9C) to the delivery arm 622, as described in more detail herein.

FIGS. 7A and 7B illustrate views of the objective housing cover 742 that may be shaped to interface with a delivery arm, such as the delivery arm 622 of FIG. 6. As an overview, the objective housing cover 742 includes a base 710 (with an upper surface 711) and a wall 712 (with an inner surface 733), which may be coupled as shown. The objective housing cover 742 has a proximal end 750 and a distal end 752. The base 710 defines an objective opening 720 and one or more light opening(s) 722 (722a and/or 722b). The wall 712 optionally includes a cover tab 730 and a cover catch 732.

The objective housing cover 742 couples to a delivery arm (e.g., the delivery arm 622 of FIG. 6) that has an objective housing (e.g., the objective housing 640 of FIG. 6). The objective housing cover 742 protects the area of the delivery arm around the objective housing from contamination from a user. The objective housing cover 742 may have any suitable size and/or shape. In the example, the objective housing cover 742 is sized and shaped to conform to the delivery arm proximate to the objective housing. For example, the upper surface 711 of the base 710 may be shaped to conform to the delivery arm. As another example, the inner surface 733 of the wall 712 may be shaped to conform to the delivery arm. The base 710 supports the wall 712 and may protect one or more parts of the delivery arm (e.g., the underside of the delivery arm) from contamination. The objective opening 720 is shaped to receive the objective housing 640 when the objective housing cover 742 is coupled to the delivery arm 622. A light opening 722 (722a or 722b) is located, sized, and shaped to allow light from a light device (e.g., light device 650a or 650b of FIG. 6) to pass through the objective housing cover 742.

The wall 712 may protect one or more parts of the delivery arm (e.g., the distal end of the delivery arm) from contamination. The wall 712 is disposed outwardly from the base 710. For example, the wall 712 may be coupled to the perimeter at the distal end 752 of the base. The wall 712 may be arranged relative to the base 710 in any suitable manner, e.g., to conform to the delivery arm. In the example, the wall 712 substantially perpendicular to the base 710, but may be at an acute angle or an obtuse angle relative to the base 710. Additionally or alternatively, the wall 712 may be curved if the outer surface of the delivery arm is curved.

The cover tab 730 may facilitate manual separation of the objective housing cover 742 from the delivery arm and may have any suitable size and/or shape. In the example, the cover tab 730 is a protrusion located at the distal end 752 of the objective housing cover 742 corresponding to the front of the delivery arm and sized and shaped to allow the user to remove the objective housing cover 742 from the delivery arm.

The cover catch 732 removably couples the objective housing cover 742 to the delivery arm and may have any suitable size and/or shape. In the example, an inner surface of the wall 712 forms a protrusion as the cover catch 732, and an outer surface of the delivery arm forms an indentation that interfaces with the cover catch 732. The protrusion and the indentation may have any suitable size and/or shape. In the example, the protrusion and the indentation are sized and shaped such that the indentation receives the protrusion to removably couple the objective housing cover 742 to the delivery arm. In other examples (not shown), the objective housing cover 742 may have one or more indentation(s) that receive one or more protrusion(s) of the delivery arm 422.

FIGS. 8A and 8B illustrate views of the objective housing cover 842. The objective housing cover 842 includes a base 810 and a wall 812. The base 810 includes an objective opening 820 and one or more light opening(s) 822 (822a to 822d). The objective housing cover 742 may be substantially similar to the objective housing cover 742 of FIGS. 7A and 7B, with the exception that the base 810 and the wall 812 are longer to cover a greater area of the delivery arm 622. The base 810 includes additional light opening(s) 822 (822c and/or 822d) and a larger objective opening 820. The objective opening 820 may include a lip 827 that runs along at least a portion of the objective opening 820. The objective housing cover 842 has a proximal end 850 and a distal end 852.

FIGS. 9A-9C illustrate views of the objective housing cover 942 that includes a tubing management clip 960. The objective housing cover 942 may be similar or comparable to the objective housing cover 824, although the objective housing cover 942 may include the tubing management clip 960. The objective housing cover 942 includes a base 910 and a wall 912, and the outer surface of the wall 912 has the tubing management clip 960 (or “clip 960”). The tubing management clip 960 may be used to secure to the wall 912 and/or otherwise manage tubing used with an ophthalmic system. For example, the clip 960 may be used to maintain sterile tubing above the work field and out of the user’s way when, e.g., a laser is used under a sterile protocol as in an operating room.

In the illustrated example, the tubing management clip 960 includes a slanted section 970, a middle section 972, and/or a secured section 974, which may be coupled as shown. The slanted section 970 forms an opening 964 of the clip 960. The secured section 974 is coupled to the outer surface of the wall 912. The middle section 972 is disposed between the slanted section 970 and the secured section 974.

The slanted section 970 may have an angled portion 980 and a lip portion 982. The angled portion 980 may guide the tubing into the clip 960 and may be at any suitable angle relative to the wall 912, such as an angle in the range of 0 to 90 degrees, e.g., 0 to 30 degrees, 30 to 60 degrees, and/or 60 to 90 degrees. The lip portion 982 may be used to hang excess tubing and may be substantially parallel to the wall 912, such as at an angle of 0 to 20 degrees (e.g., 0 to 10 degrees and/or 10 to 20 degrees) relative to the wall 912.

The middle section 972 may guide the tubing into the clip 960 and may be substantially parallel to the wall 912. In one or more embodiments, middle section 972 may be a tapered section, that is, the clearance between the middle section 972 and the wall 912 may decrease from, e.g., a maximum size closer to the opening of the clip 960 to a minimum size farther from the opening of the clip 960. In an example, the maximum size may be greater than the outer diameter of the tubing (e.g., by 0.1 to 5 millimeters), and the minimum size may be less than the outer diameter of the tubing (e.g., by 0.1 to 5 millimeters). In the embodiments, as the clearance tapers, the middle section 972 compresses the tubing slightly and causes friction to allow the tubing to remain in place.

The secured section 974 may allow the tubing to move along a longitudinal axis of the tubing while remaining secured to the wall 912. In one or more embodiments, the secured section 974 may have a clearance with a size and/or shape that matches or is slightly larger than (e.g., by 0.1 to 5 millimeters) the cross-section of the tubing. In the illustrated example, the clearance is circular to accommodate tubing with a circular cross-section, but in other examples the clearance may be oval or other suitable shape to accommodate tubing with a cross-section that is an oval or other suitable shape.

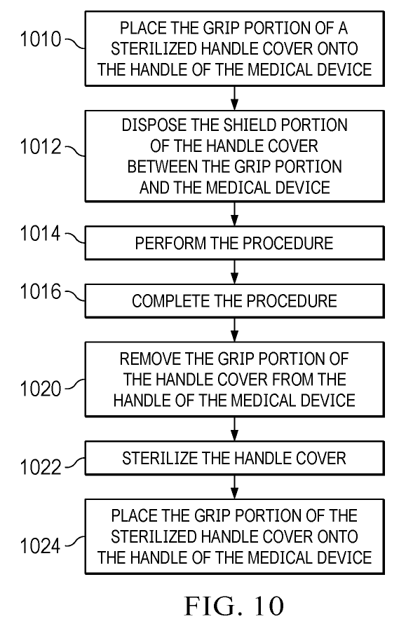

FIG. 10 illustrates an example of a method of using a handle cover, according to at least one embodiment described in the present disclosure. In the example, the handle cover has a grip portion and a shield portion and may be used for a handle of a delivery arm of a medical device during a medical procedure on a patient’s eye. The handle cover allows a user (e.g., a surgeon or other medical professional) to move the delivery arm to the proper position relative to the patient’s eye, while protecting the handle from contamination.

At step 1010, the grip portion of the handle cover, which has been sterilized, is placed onto the handle. In certain embodiments, one or more sterilized joystick covers and/or a sterilized objective housing cover may also be placed on the medical device. In some embodiments, one or more cover tabs may be used to facilitate pulling the handle cover over the handle.

At step 1012, a shield portion of the handle cover is disposed between the grip portion and the medical device. The shield portion may operate as a barrier between the user’s hand and the distal portion of the delivery arm. In one or more embodiments, a cover catch may be used to removably secure the handle cover to the handle. For example, the inner surface of the handle cover may have a protrusion, and the outer surface of the handle may have an indentation that is sized and/or shaped to receive the protrusion. As another example, the outer surface of the handle may have protrusion, and the inner surface of the handle cover may have an indentation that is sized and/or shaped to receive the protrusion.

At step 1014, the medical procedure is performed. The medical procedure may be a surgical procedure or a diagnostic procedure. For example, the medical procedure may be a laser surgical procedure.

At step 1016, the medical procedure is completed.

At step 1020, the grip portion of the handle cover is removed from the handle of the medical device. In one or more embodiments, one or more cover tabs may be used to facilitate removal of the grip portion from the handle. For example, the cover tab may be a protrusion that the user can grasp with, e.g., a thumb and a finger. In certain embodiments, one or more joystick covers and/or an objective housing cover may be removed on the medical device.

At step 1022, the handle cover is sterilized. The handle cover may be sterilizable, e.g., in an autoclave or with a disinfectant or other sterilizing agent. In certain embodiments, one or more joystick covers and/or an objective housing cover may be sterilized.

At step 1024, the grip portion of the sterilized handle cover is placed onto the handle of the medical device for the next medical procedure. In certain embodiments, the one or more sterilized joystick covers and/or the sterilized objective housing cover may be placed on the medical device for the next medical procedure. A further medical procedure may then be performed with the same handle cover as used in the initial procedure, having been sterilized between the two procedures.

The present disclosure (including the specification, claims, and drawings) includes example embodiments that are intended to aid the reader in understanding the invention and concepts contributed by the inventor to furthering the art and to enable any person skilled in the art to make or use the disclosed embodiments. Modifications (e.g., changes, substitutions, additions, omissions, and/or other modifications) to the embodiments will be readily apparent to those skilled in the art. Accordingly, modifications may be made to the embodiments without departing from the essence of the present disclosure.

In certain instances, modifications may be made to the systems disclosed herein, as apparent to those skilled in the art. For example, parts of a system may be integrated or separated, or an operation of a system may be performed by more, fewer, or other parts. In certain instances, modifications may be made to the methods disclosed herein, as apparent to those skilled in the art. For example, the methods may include more, fewer, or other operations. As another example, certain operations may be optional, combined into fewer operations, or expanded into additional operations. As yet another example, certain operations may be performed in any suitable order or simultaneously.

Furthermore, those skilled in the art will recognize that the present disclosure is not intended to be limited to the example embodiments and that the language of the disclosure is to be accorded the widest scope consistent with the present disclosure. Terms (which may include one or more words) that describe inclusion are generally intended as “open” terms in that they generally do not imply exclusion. For example, the term “including” may be interpreted as “including, but not limited to” or “including at least”; the term “having” may be interpreted as “having, but not limited to” or “having at least”; and the term “comprising” may be interpreted as “comprising, but not limited to” or “comprising at least”, etc.

Additionally, if a specific number is intended, such intent will be explicitly recited in the claim. In the absence of the explicit recitation of a specific number, no such intent is present. If a specific number is explicitly recited, such recitation should be interpreted to mean at least the recited number. For example, the bare recitation of “two Xs”, without other modifiers, may mean “at least two Xs” or “two or more Xs”. Moreover, the use of an indefinite article (e.g., “a” or “an”) or definite article (e.g., “the”) to introduce a noun phrase should not be construed to limit the noun phrase to one, but may be interpreted as an open term “at least one” or “one or more”. This holds even when the same claim includes an open term (e.g., “one or more” or “at least one”) and an indefinite or definite article (e.g., “a” or “an” or “the”).

Moreover, a selection from a list of items should be understood to contemplate a selection of any suitable individual item or any suitable combination of items. For example, the general construction “at least one of A, B, and C” or “one or more of A, B, and C” may include A alone; B alone; C alone; A and B together; A and C together; B and C together; and A, B, and C together. Moreover, any disjunctive term presenting two or more alternative items may be understood to contemplate including one of the items, either of the items, or both items. For example, the general construction “A or B” or “A and/or B” may include A alone, B alone, and A and B together. Additionally, the use of the terms “first,” “second,” “third,” etc. are not necessarily used herein to connote a specific order. For example, the terms “first,” “second,” “third,” etc., may be used to distinguish between different elements.

Additionally, relative terms may be used and understood as typically used in the relevant art. By way of example, relative terms such as “approximately,” “substantially,” “about,” “roughly,” or other similar relative terms mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When such relative terms are used to refer to numbers or values that are part of a range, the term can be used to modify both the minimum and maximum numbers or values.

To aid the Patent Office and readers in interpreting the claims, Applicants note that they do not intend any of the claims or claim elements to invoke 35 U.S.C. §112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the Applicants to refer to structures known to those skilled in the art and is not intended to invoke 35 U.S.C. §112(f).

What is claimed is:

1. A handle cover for a handle of a medical device, the handle cover comprising:
a grip portion configured to receive the handle of the medical device, the grip portion having:
a proximal end and a distal end;
an inner surface shaped to receive to the handle of the medical device, the inner surface forming an opening at the proximal end, the opening configured to receive the handle of the medical device; and
an outer surface configured to facilitate a manual manipulation of the handle of the medical device; and
a shield portion coupled to the grip portion, the shield portion disposed between the grip portion and the medical device when the grip portion has received the handle of the medical device.

2. The handle cover of claim 1, the grip portion comprising one or more button pads, a button pad of the one or more button pads configured to allow for a manual operation of a button of the handle of the medical device.

3. The handle cover of claim 1, the handle cover comprising a cover catch configured to removably secure the handle cover to the handle of the medical device.

4. The handle cover of claim 1, the handle cover comprising a cover tab configured to allow for a manual separation of the handle cover from the handle of the medical device.

5. The handle cover of claim 1, the shield portion coupled to the proximal end of the grip portion.

6. The handle cover of claim 1, the shield portion coupled to the distal end of the grip portion.

7. The handle cover of claim 1, a grip portion length of the grip portion being within four centimeters of a handle length of the handle.

8. The handle cover of claim 1, a shield length of the shield being within four centimeters of a grip length of the grip portion.

9. The handle cover of claim 1, comprising a silicone material.

10. A method of utilizing a handle cover for a handle of a medical device, comprising:
placing a grip portion of the handle cover onto the handle of the medical device for a first medical procedure, the grip portion comprising a proximal end and a distal end, the grip portion having an inner surface shaped to receive the handle of the medical device, the inner surface forming an opening at the proximal end, the opening configured to receive the handle of the medical device, the grip portion having an outer surface configured to facilitate a manual manipulation of the handle of the medical device;
disposing a shield portion of the handle cover between the grip portion and the medical device, the shield portion coupled to the grip portion;
removing the grip portion of the handle cover from the handle of the medical device to remove the handle cover from the medical device after the first medical procedure;
sterilizing the handle cover; and
placing the grip portion of the handle cover onto the handle of the medical device for a second medical procedure.

11. The method of utilizing the handle cover of claim 10, the placing the grip portion of the handle cover onto the handle of the medical device for the first medical procedure comprising:
removably securing the handle cover to the handle of the medical device using a cover catch of the handle cover.

12. The method of utilizing the handle cover of claim 10, the removing the grip portion of the handle cover from the handle of the medical device to remove the handle cover from the medical device comprising:
removing the grip portion of the handle cover from the handle of the medical device using a cover tab of the handle cover.

13. An objective housing cover for an objective housing of a delivery arm of a medical device, the objective housing cover comprising:
a base having:
a proximal end and a distal end;
an upper surface shaped to conform to the objective housing of the delivery arm of the medical device; and
an objective opening defined by the proximal end of the base, the objective opening shaped to receive the objective housing of the delivery arm; and
a wall disposed outwardly from the base, the wall proximate to the distal end of the base, an inner surface of the wall shaped to conform to the delivery arm.

14. The objective housing cover of claim 13, the wall substantially perpendicular to the base.

15. The objective housing cover of claim 13, the base defining one or more light openings, a light opening of the one or more light openings configured to allow a light from the delivery arm to pass through the base.

16. The objective housing cover of claim 13, the distal end of the base comprising a cover tab, the cover tab configured to facilitate manual removal of the objective housing cover from the delivery arm.

17. The objective housing cover of claim 13, the inner surface of the wall comprising a cover catch, the cover catch configured to couple with a delivery arm catch to removably couple the objective housing cover to the delivery arm.

18. The objective housing cover of claim 13, an outer surface of the wall having a tubing management clip configured to couple a tubing to the wall.

19. The objective housing cover of claim 18, the tubing management clip comprising a slanted section configured to:
form an opening of the tubing management clip; and
guide the tubing into the clip.

20. The objective housing cover of claim 18, the tubing management clip comprising a middle section having a clearance relative to the wall, the clearance having:
a maximum size closer to an opening of the tubing management clip; and
a minimum size farther from the opening of the tubing management clip.

21. The objective housing cover of claim 18, the tubing management clip comprising a secured section configured to allow the tubing to move in a direction along an axis of the tubing.

22. A set of a plurality of manual controller covers for a medical device, comprising:
a handle cover for a handle of the medical device;
at least one joystick cover for at least one joystick of the medical device; and
an objective housing cover for an objective housing of the medical device.

23. The set of the plurality of manual controller covers of claim 22, the handle cover comprising:
a grip portion configured to receive the handle of the medical device; and
a shield portion coupled to the grip portion, the shield portion disposed between the grip portion and the medical device when the grip portion has received the handle of the medical device.

24. The set of the plurality of manual controller covers of claim 22, the objective housing cover comprising:
a base having an objective opening defined by a proximal end of the base, the objective opening shaped to receive the objective housing; and
a wall disposed outwardly from the base, the wall proximate to a distal end of the base.

25. The set of the plurality of manual controller covers of claim 22, the at least one joystick cover comprising:
a base having an annular shape with an inner ring shaped to receive the at least one joystick; and
a wall disposed outwardly from the base, the wall having an inner surface and an outer surface, the inner surface shaped to receive the at least one joystick, the outer surface having a texture that facilitates a manual manipulation of the at least one joystick.

26. The set of manual controller covers of claim 22, further comprising a container configured to hold the plurality of manual controller covers.
